(19) United States
(12) Patent Application Publication
(10) Pub. No.: US 2026/0263161 A1
(43) Pub. Date: Sep. 10, 2026

(54) VIDEO-BASED NAVIGATION FOR GUIDING A TOOL TOWARDS A TARGET

(71) Applicants: Smith & Nephew, Inc., Memphis, TN (US); Smith & Nephew Asia Pacific Pte. Limited, Singapore (SG)

(72) Inventors: Brian William Quist, Salem, NH (US); Nicholas Ryan Labriola, Medway, MA (US); Rafal Z. Jezierski, Candia, NH (US); Long Tran, Boston, MA (US)

(73) Assignees: Smith & Nephew, Inc., Memphis, TN (US); Smith & Nephew Asia Pacific Pte. Limited, Singapore (SG)

(21) Appl. No.: 19/561,160

(22) Filed: Mar. 9, 2026

Related U.S. Application Data
(60) Provisional application No. 63/769,259, filed on Mar. 10, 2025.

(51) Int. Cl. A61B 34/20; A61B 34/10; A61B 90/00

(52) U.S. Cl. CPC A61B 34/20; A61B 34/10; A61B 90/37; A61B 2034/102; A61B 2034/105; A61B 2034/2065; A61B 2034/2068; A61B 2090/363

(57) ABSTRACT

A processor is configured to execute instructions stored in memory to control a surgical navigation system. Executing the instructions causes the processor to control the surgical navigation system to receive one or more images of a surgical environment, determine a plurality of points on patient anatomy indicated by an instrument identified in the surgical environment, define a bounding plane on the patient anatomy based on the plurality of points, and generate, for display, visual guidance based on the bounding plane and a location of the instrument. The visual guidance includes at least one constraint for a location of a tunnel to be formed in the patient anatomy, a visual indicator of the location of the tunnel based on the location of the instrument, and an indication of whether the location of the tunnel satisfies the at least one constraint.

### CROSS-REFERENCE TO RELATED APPLICATIONS

This application claims the benefit of U.S. Provisional Application No. 63/769,259, filed on March 10, 2025. The entire disclosure of the application referenced above is incorporated herein by reference.

### FIELD

The present disclosure relates to instruments or tools for surgical navigation systems, and more particularly to providing navigation guidance for instruments.

### BACKGROUND

The background description provided here is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Arthroscopic surgical procedures are minimally invasive surgical procedures in which access to the surgical site within the body is by way of small keyholes or ports through the patient’s skin. The various tissues within the surgical site are visualized by way of an arthroscope placed through a port, and the internal scene is shown on an external display device. The tissue may be repaired or replaced through the same or additional ports. In computer-assisted surgical procedures (e.g., replacement of the anterior cruciate ligament (ACL), reduction of femora-acetabular impingement), the location of various objects within the surgical site may be determined relative to the bone by way of images captured by an arthroscope and a three-dimensional model of the bone.

### SUMMARY

A processor is configured to execute instructions stored in memory to control a surgical navigation system. Executing the instructions causes the processor to control the surgical navigation system to receive one or more images of a surgical environment, determine a plurality of points on patient anatomy indicated by an instrument identified in the surgical environment, define a bounding plane on the patient anatomy based on the plurality of points, and generate, for display, visual guidance based on the bounding plane and a location of the instrument. The visual guidance includes at least one constraint for a location of a tunnel to be formed in the patient anatomy, a visual indicator of the location of the tunnel based on the location of the instrument, and an indication of whether the location of the tunnel satisfies the at least one constraint.

In other features, at least one constraint includes a distance from the location of the tunnel to a feature of the patient anatomy. The distance is a distance to a back wall of a lateral femoral condyle. The at least one constraint includes a distance percentage along an axis defined by a posterior reference point on a lateral femoral condyle and an anterior-most point of a condylar wall. The indication of whether the location of the tunnel satisfies the at least one constraint includes a characteristic of display of the at least one constraint.

In other features, executing the instructions causes the processor to control the surgical navigation system to receive one or more parameters and display the visual guidance based on the one or more parameters. The one or more parameters include at least one of a femoral entry point tunnel diameter and a target back wall thickness. The plurality of points includes at least one of a posterior reference point on a femoral condyle and an anterior-most point of a condylar wall. The plurality of points further includes one or more points along a surface of an inferior condyle wall between the posterior reference point and the anterior-most point. The plurality of points further includes a superior-most point of the bounding plane.

A method for controlling a surgical navigation system includes receiving one or more images of a surgical environment, determining a plurality of points on patient anatomy indicated by an instrument identified in the surgical environment, defining a bounding plane on the patient anatomy based on the plurality of points, and generating, for display, visual guidance based on the bounding plane and a location of the instrument. The visual guidance includes at least one constraint for a location of a tunnel to be formed in the patient anatomy, a visual indicator of the location of the tunnel based on the location of the instrument, and an indication of whether the location of the tunnel satisfies the at least one constraint.

In other features, the at least one constraint includes a distance from the location of the tunnel to a feature of the patient anatomy. The distance is a distance to a back wall of a lateral femoral condyle. The at least one constraint includes a distance percentage along an axis defined by a posterior reference point on a lateral femoral condyle and an anterior-most point of a condylar wall. The indication of whether the location of the tunnel satisfies the at least one constraint includes a characteristic of display of the at least one constraint.

In other features, the method further includes receiving one or more parameters and displaying the visual guidance based on the one or more parameters. The one or more parameters include at least one of a femoral entry point tunnel diameter and a target back wall thickness. The plurality of points includes at least one of a posterior reference point on a femoral condyle and an anterior-most point of a condylar wall. The plurality of points further includes one or more points along a surface of an inferior condyle wall between the posterior reference point and the anterior-most point and a superior-most point of the bounding plane.

A surgical navigation system includes an image capture device configured to capture one or more images of a surgical environment and a controller. The controller is configured to detect a location of an instrument within the surgical environment using the one or more images and one or more fiducial markers on the instrument, identify a plurality of points on patient anatomy based on respective locations of the instrument within the surgical environment, define a bounding plane on the patient anatomy based on the plurality of points, and generate, for display, visual guidance based on the bounding plane and the location of the instrument. The visual guidance includes at least one constraint for a location of a tunnel to be formed in the patient anatomy, a visual indicator of the location of the tunnel based on the location of the instrument, and an indication of whether the location of the tunnel satisfies the at least one constraint. The at least one constraint includes at least one of a distance from an edge of the tunnel to a back wall of a lateral femoral condyle and a distance percentage along an axis defined by a posterior reference point on the lateral femoral condyle and an anterior-most point of a condylar wall.

Further areas of applicability of the present disclosure will become apparent from the detailed description, the claims and the drawings. The detailed description and specific examples are intended for purposes of illustration only and are not intended to limit the scope of the disclosure.

### BRIEF DESCRIPTION OF THE DRAWINGS

The present disclosure will become more fully understood from the detailed description and the accompanying drawings, wherein:

FIG. 1 shows a surgical system in accordance with at least some embodiments;

FIG. 2 shows a conceptual drawing of a surgical site with various objects within the surgical site tracked, in accordance with at least some embodiments;

FIG. 3 shows a method in accordance with at least some embodiments;

FIG. 4 is an example video display showing portions of a femur and a bone fiducial during a registration procedure, in accordance with at least some embodiments;

FIG. 5 shows a method in accordance with at least some embodiments;

FIGS. 6A and 6B show example probes used in surgical navigation systems;

FIGS. 7A and 7B show an example awl used in surgical navigation systems in accordance with at least some embodiments;

FIGS. 8A, 8B, 8C, 8D, 8E, 8F, and 8G show an example workflow/navigation process performed to select/identify points on patient anatomy for tunnel positioning in accordance with at least some embodiments;

FIGS. 9A and 9B show example visual guidance provided by the surgical navigation system in accordance with at least some embodiments;

FIGS. 10A and 10B show examples of visual guidance displayed to assist a user to locate a tunnel in accordance with at least some embodiments;

FIG. 11 illustrates steps of an example method for provide navigation guidance for locating a tunnel in accordance with at least some embodiments; and

FIG. 12 shows an example computer system in accordance with at least some embodiments.

In the drawings, reference numbers may be reused to identify similar and/or identical elements.

### DEFINITIONS

Various terms are used to refer to particular system components. Different companies may refer to a component by different names – this document does not intend to distinguish between components that differ in name but not function. In the following discussion and in the claims, the terms “including” and “comprising” are used in an open-ended fashion, and thus should be interpreted to mean “including, but not limited to… .” Also, the term “couple” or “couples” is intended to mean either an indirect or direct connection. Thus, if a first device couples to a second device, that connection may be through a direct connection or through an indirect connection via other devices and connections.

An endoscope having “a single optical path” through an endoscope shall mean that the endoscope is not a stereoscopic endoscope having two distinct optical paths separated by an interocular distance at the light collecting end of the endoscope. The fact that an endoscope has two or more optical members (e.g., glass rods, optical fibers) forming a single optical path shall not obviate the status as a single optical path.

Similarly, spatial and functional relationships between elements (for example, between device, modules, circuit elements, etc.) are described using various terms, including “connected,” “engaged,” “coupled,” “adjacent,” “next to,” “on top of,” “above,” “below,” and “disposed.” Unless explicitly described as being “direct,” when a relationship between first and second elements is described in the above disclosure, that relationship can be a direct relationship where no other intervening elements are present between the first and second elements, but can also be an indirect relationship where one or more intervening elements are present (either spatially or functionally) between the first and second elements. Nevertheless, this paragraph shall serve as antecedent basis in the claims for referencing any electrical connection as “directly coupled” for electrical connections shown in the drawing with no intervening element(s).

Terms of degree, such as “substantially” or “approximately,” are understood by those skilled in the art to refer to reasonable ranges around and including the given value and ranges outside the given value, for example, general tolerances associated with manufacturing, assembly, and use of the embodiments. The term “substantially,” when referring to a structure or characteristic, includes the characteristic that is mostly or entirely present in the characteristic or structure. As one example, numerical values that are described as “approximate” or “approximately” as used herein may refer to a value within +/- 5% of the stated value.

“A”, “an”, and “the” as used herein refers to both singular and plural referents unless the context clearly dictates otherwise. By way of example, “a processor” programmed to perform various functions refers to one processor programmed to perform each and every function, or more than one processor collectively programmed to perform each of the various functions. To be clear, an initial reference to “a [referent]”, and then a later reference for antecedent basis purposes to “the [referent]”, shall not obviate the fact the recited referent may be plural.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context. As used herein, the phrase at least one of A, B, and C should be construed to mean a logical (A OR B OR C), using a non-exclusive logical OR, and should not be construed to mean “at least one of A, at least one of B, and at least one of C.”

The terms “input” and “output” when used as nouns refer to connections (e.g., electrical, software) and/or signals, and shall not be read as verbs requiring action. For example, a timer circuit may define a clock output. The example timer circuit may create or drive a clock signal on the clock output. In systems implemented directly in hardware (e.g., on a semiconductor substrate), these “inputs” and “outputs” define electrical connections and/or signals transmitted or received by those connections. In systems implemented in software, these “inputs” and “outputs” define parameters read by or written by, respectively, the instructions implementing the function. In examples where used in the context of user input, “input” may refer to actions of a user, interactions with input devices or interfaces by the user, etc.

“Controller,” “module,” or “circuitry” shall mean, alone or in combination, individual circuit components, an application specific integrated circuit (ASIC), a microcontroller with controlling software, a reduced-instruction-set computer (RISC) with controlling software, a digital signal processor (DSP), a processor with controlling software, a programmable logic device (PLD), a field programmable gate array (FPGA), or a programmable system-on-a-chip (PSOC), configured to read inputs and drive outputs responsive to the inputs.

As used to describe various surgical instruments or devices, such as a probe, the term “proximal” refers to a point or direction nearest a handle of the probe (e.g., a direction opposite the probe tip). Conversely, the term “distal” refers to a point or direction nearest the probe tip (e.g., a direction opposite the handle).

For the purposes of this disclosure, a non-transitory computer readable medium (or computer-readable storage medium/media) stores computer data, which data can include computer program code (or computer-executable instructions) that is executable by a computer, in machine-readable form. By way of example, and not limitation, a computer readable medium may comprise computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure, the term “server” should be understood to refer to a service point that provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure, a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire‐line type connections, wireless type connections, cellular or any combination thereof. Likewise, sub‐networks, which may employ differing architectures or may be compliant or compatible with differing protocols, may interoperate within a larger network.

For purposes of this disclosure, a “wireless network” should be understood to couple client devices with a network. A wireless network may employ stand‐alone ad‐hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router (WR) mesh, or 2nd, 3rd, 4th or 5th generation (2G, 3G, 4G, or 5G) cellular technology, mobile edge computing (MEC), Bluetooth, 802.11b/g/n, or the like. Network access technologies may enable wide area coverage for devices, such as client devices with varying degrees of mobility, for example. In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack‐mounted servers, desktop computers, laptop computers, set top boxes, integrated devices combining various features, such as two or more features of the foregoing devices, or the like.

For purposes of this disclosure, a client (or consumer or user) device, referred to as user equipment (UE)), may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

In some embodiments, as discussed below, the client device can also be, or can communicatively be coupled to, any type of known or to be known medical device (e.g., any type of Class I, II or III medical device), such as, but not limited to, a MRI machine, CT scanner, Electrocardiogram (ECG or EKG) device, photopletismograph (PPG), Doppler and transmit-time flow meter, laser Doppler, an endoscopic device neuromodulation device, a neurostimulation device, and the like, or some combination thereof.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

Various examples are directed to methods and systems of registering a three-dimensional (3D) model of a rigid structure, such as bone. More particularly, various examples are directed to methods and related systems for identifying surface features of a rigid structure visible in a video stream, and using the surface features to register a three-dimensional model for use in computer-assisted navigation of the surgical procedure (e.g., Computer-Aided Surgery, or CAS). In some examples, the surface features are determined using touchless techniques based on a known or calculated motion of the camera. In other examples, the surface features are gathered using a touch probe that is not itself directly tracked; rather, the pose of the touch probe, and thus the locations of the distal tip of the touch probe touching the bone, may be determined by segmenting the frames of the video stream and pose estimation. In yet still further examples, the three-dimensional model may be registered by use of a patient-specific instrument that couples to the rigid structure in only one orientation; thus, a fiducial coupled to the patient-specific instrument, or in some cases the patient-specific instrument itself without a fiducial, may be used to register a three-dimensional anatomical (e.g., bone) model.

Video-based surgical navigation (VBSN) techniques that use patient-specific instruments may include using visual fiducials or fiducial markers (also called visual markers) attached to patient anatomy to guide the surgeon throughout the medical procedure. The video-based navigation process requires precise registration of a pre-operative anatomical model with data acquired intra-operatively. The registration process or procedure requires the surgeon to digitize the surface of interest that corresponds to the pre-operative model. The fiducial markers with known visual patterns that are attached to the anatomies define reference frames to which the pre-operative model and the intra-operative acquired data are aligned. The fiducial markers may be attached both to the targeted anatomy and to the instruments and subsequently tracked such that their relative poses can be accurately estimated (e.g., by applying 3D computer vision methods on the images/video acquired by a camera). These relative poses allow the instruments to be located with respect to the anatomy at every frame time instant. For example, VBSN facilitates the tracking of instruments with respect to the targeted anatomy to which a fiducial is rigidly attached (which may be referred to as a “base marker”).

In some examples of a registration procedure, a user probes an anatomical surface using a handheld probe. Collected points (e.g., a point cloud) are processed (e.g., using a machine learning algorithm) and matched to a bone model, such as a bone model created via a scan (e.g., a CT or MRI scan) or other technique. For example, the bone model is overlaid on top of a live arthroscopic video feed to provide an augmented or mixed reality visual representation of a surgical or anatomical site.

In this manner, by using a probe with a calibrated tip (i.e., a probe that is calibrated such that the 3D position of the tip of the probe relative to the reference frame of the tip is known), 3D points can be intra-operatively reconstructed in the reference frame of the fiducial marker. Once the tip is in contact with the surface of patient anatomy, the set of reconstructed 3D points is a representation of the anatomy of interest that is provided as input to the registration procedure.

Various procedures, such as anterior-cruciate ligament (ACL) repair/reconstruction, include positioning and forming of tunnels in patient anatomy (e.g., femoral and/or tibial tunnels). Improper tunnel position (“malposition”) can lead to ACL reconstruction failure. As one example, malposition can result in the femoral tunnel being too close to the posterior cortex of the femur. In some examples, positioning and planning of the femoral tunnel is performed with direct visualization. Mechanical tools (e.g., offset guides) may be used to facilitate planning. In other examples, a surgical, video-based navigation system can be used to facilitate tunnel positioning (e.g., using external IR tracking arrays).

Surgical navigation systems and methods according to the present disclosure use a camera (e.g., an arthroscopic camera) to identify and locate a marker fixed to patient anatomy (e.g., a fiducial marker affixed to bone). The system is configured to receive one or more inputs (e.g., user inputs) identifying anatomical boundaries or landmarks within a surgical environment and provide navigation guidance to a target (e.g., a target tunnel position) based on the inputs and various other operational parameters/settings.

For example, a surgical instrument or tool, such as an awl, can be used to form an indentation or other feature in the bone, such as by using a mallet or other tool to apply force to the awl. In accordance with the principles of the present disclosure, the awl may include fiducial markers or markings. With the fixed fiducial marker installed and the fiducial markers on the awl, the awl can be tracked while navigating between and/or designating various points on patient anatomy in accordance with various settings and guidance provided by the surgical navigation system as described below in more detail. Although described herein with respect to an awl, the principles of the present disclosure may be implemented with other types of surgical instruments, such as probes. Further, although described with respect to fixed fiducial markers installed in patient anatomy, in some examples the principles of the present disclosure may be implemented without the used of installed (e.g., bone) fiducial markers, such as by using other fixed landmarks or anatomical points.

Various examples described herein relate to ACL repair (e.g., for placing femoral and/or tibial tunnels during ACL reconstruction), and thus the discussion below is based on the developmental context. In this context, the rigid structure is bone and/or cartilage, and the three-dimensional model is a three-dimensional bone model. However, the techniques described herein are applicable to any suitable rigid anatomical structure, such as teeth. Moreover, the various techniques may be applicable to many types of surgical procedures, such as repairs associated with the knee, the hip, the shoulder, the wrist, or the ankle. The techniques may be applicable not only to ligament repair (e.g., medial collateral ligament repair, lateral collateral ligament repair, and posterior cruciate ligament repair), but also for planning and placing anchors to reattach soft tissue (e.g., reattaching the labrum of the hip, the rotator cuff, or the meniscal root), and surgical procedures to address femoroacetabular impingement. Thus, the description and developmental context shall not be read as a limitation of the applicability of the teachings.

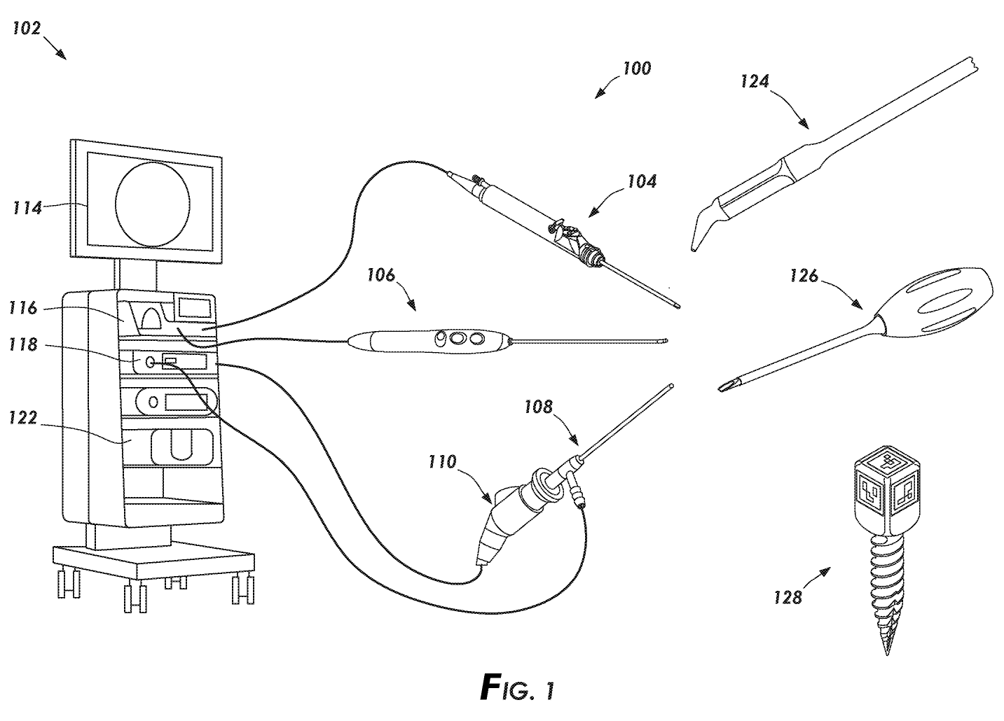

FIG. 1 shows a surgical system (not to scale) 100 in accordance with at least some embodiments. In particular, the example surgical system 100 comprises a tower or device cart 102, an example mechanical resection instrument 104, an example plasma-based ablation instrument (hereafter just ablation instrument 106), and an endoscope in the example form of an arthroscope 108 and attached camera head or camera 110. In the example systems, the arthroscope 108 is a rigid device, unlike endoscopes for other procedures, such as upper-endoscopies. The device cart 102 may comprise a display device 114, a resection controller 116, and a camera control unit (CCU) together with an endoscopic light source and video controller 118. In example cases the combined CCU and video controller 118 not only provides light to the arthroscope 108 and displays images received from the camera 110, but also implements various additional aspects, such as registering a three-dimensional bone model with the bone visible in the video images, and providing computer-assisted navigation during the surgery. Thus, the combined CCU and video controller are hereafter referred to as surgical controller 118. In other cases, however, the CCU and video controller may be a separate and distinct system from the controller that handles registration and computer-assisted navigation, yet the separate devices would nevertheless be operationally coupled.

The example device cart 102 further includes a pump controller 122 (e.g., single or dual peristaltic pump). Fluidic connections of the mechanical resection instrument 104 and ablation instrument 106 to the pump controller 122 are not shown so as not to unduly complicate the figure. Similarly, fluidic connections between the pump controller 122 and the patient are not shown so as not to unduly complicate the figure. In the example system, both the mechanical resection instrument 104 and the ablation instrument 106 are coupled to the resection controller 116 being a dual-function controller. In other cases, however, there may be a mechanical resection controller separate and distinct from an ablation controller. The example devices and controllers associated with the device cart 102 are merely examples, and other examples include vacuum pumps, patient-positioning systems, robotic arms holding various instruments, ultrasonic cutting devices and related controllers, patient-positioning controllers, and robotic surgical systems.

FIG. 1 further shows additional instruments that may be present during an arthroscopic surgical procedure. In particular, FIG. 1 shows an example touch probe 124, a drill guide or aimer 126, and a bone fiducial 128. The touch probe 124 may be used during the surgical procedure to provide information to the surgical controller 118, such as information to register a three-dimensional bone model to an underlying bone visible in images captured by the arthroscope 108 and camera head 110. The aimer 126 may be used as a guide for placement and drilling with a drill wire to create an initial or pilot tunnel through the bone. The bone fiducial 128 may be affixed or rigidly attached to the bone and serve as an anchor location for the surgical controller 118 to know the orientation of the bone (e.g., after registration of a three-dimensional bone model). Additional tools and instruments will be present, such as the drill wire, various reamers for creating the throughbore and counterbore aspects of a tunnel through the bone, and various tools, such as for suturing and anchoring a graft. These additional tools and instruments are not shown so as not to further complicate the figure. The specification now turns to a workflow for an example anterior cruciate ligament repair.

A surgical procedure may begin with a planning phase. The example anterior cruciate ligament repair may start with imaging (e.g., X-ray imaging, computed tomography (CT), magnetic resonance imaging (MRI)) of the knee of the patient, including the relevant anatomy like the lower portion of the femur, the upper portion of the tibia, and the articular cartilage. The imaging may be preoperative imaging, hours or days before the intraoperative repair, or the imaging may take place within the surgical setting just prior to the intraoperative repair. The discussion that follows assumes MRI imaging, but again many different types of imaging may be used. The image slices from the MRI imaging can be segmented such that a volumetric model or three-dimensional model of the anatomy is created. Any suitable currently available, or after developed, segmentation technology may be used to create the three-dimensional model. More specifically to the example of anterior cruciate ligament repair, a three-dimensional bone model of the lower portion of the femur, including the femoral condyles, is created.

Using the three-dimensional bone model, an operative plan is created that comprises choosing a planned-tunnel path through the femur, including locations of the apertures of the bone that define the ends of the tunnel. For an example inside-out repair, the aperture within the femoral notch is the entry location for the drilling, and the aperture on the lateral surface of the femur is the exit location. For an outside-in repair, the entry and exit locations for drilling are swapped. Still assuming an inside-out repair, the entry location may be selected to be the same as, or close to, the attachment location of the native anterior cruciate ligament to the femur within the femoral notch. In some cases, selecting the entry location within the femoral notch may involve use of a Bernard & Hertel Quadrant or grid placed on a fluoroscopic image, or by placing the Bernard & Hertel Quadrant on a simulated fluoroscopic image created from the three-dimensional bone model. Based on use of the Bernard & Hertel Quadrant, an entry location for the tunnel is selected. For an inside-out repair, selection of the exit location is less restrictive, not only because the portion of the tunnel proximate to the exit location is used for placement of the anchor for the graft, but also because the exit location is approximately centered in the femur (considered anteriorly to posteriorly), and thus issues of bone wall thickness at the exit location are of less concern. In some cases, a three-dimensional bone model of the proximal end of the tibia is also created, and the surgeon may likewise choose planned-tunnel path(s) through the tibia.

The results of the planning may include: a three-dimensional bone model of the distal end of the femur; a three-dimensional bone model for a proximal end of the tibia; an entry location and exit location through the femur and thus a planned-tunnel path for the femur; and an entry location and exit location through the tibia and thus a planned-tunnel path through the tibia. Other surgical parameters may also be selected during the planning, such as tunnel throughbore diameters, tunnel counterbore diameters and depth, desired post-repair flexion, and the like, but those additional surgical parameters are omitted so as not to unduly complicate the specification.

The specification now turns to intraoperative aspects. The intraoperative aspects include steps and procedures for setting up the surgical system to perform the various repairs. It is noted, however, that some of the intraoperative aspects (e.g., optical system calibration), may take place before any ports or incisions are made through the patient’s skin, and in fact before the patient is wheeled into the surgical room. Nevertheless, such steps and procedures may be considered intraoperative as they take place in the surgical setting and with the surgical equipment and instruments used to perform the actual repair.

The example ACL repair is conducted arthroscopically and is computer-assisted in the sense the surgical controller 118 is used for arthroscopic navigation within the surgical site. More particularly, in example systems the surgical controller 118 provides computer-assisted navigation during the ligament repair by tracking location of various objects within the surgical site, such as the location of the bone within the three-dimensional coordinate space of the view of the arthroscope, and location of the various instruments (e.g., a drill wire) within the three-dimensional coordinate space of the view of the arthroscope. The specification turns to brief description of such tracking techniques.

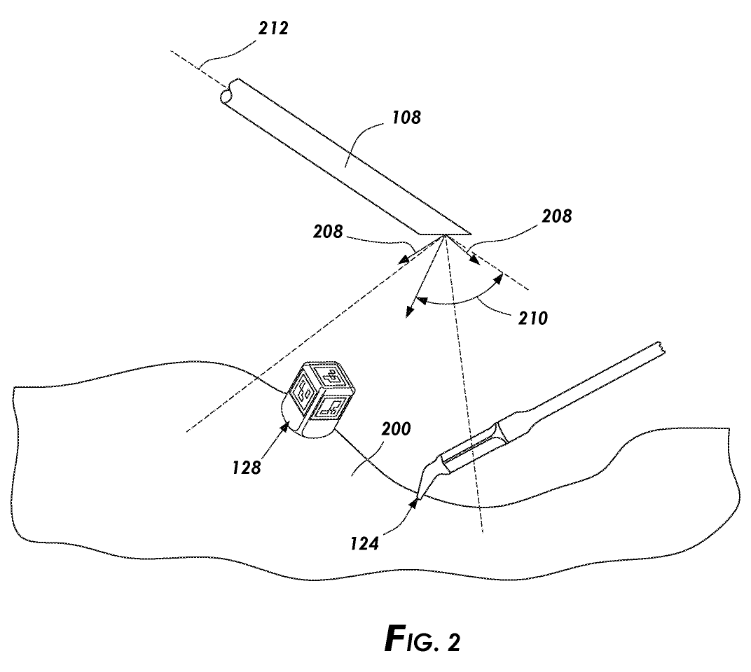

FIG. 2 shows a conceptual drawing of a surgical site with various objects within the surgical site. In particular, visible in FIG. 2 is a distal end of the arthroscope 108, a portion of a bone 200 (e.g., femur), the bone fiducial 128 within the surgical site, and the touch probe 124. Each is addressed in turn.

The arthroscope 108 illuminates the surgical site with visible light. In the example of FIG. 2, the illumination is illustrated by arrows 208. The illumination provided to the surgical site is reflected by various objects and tissues within the surgical site, and the reflected light that returns to the distal end enters the arthroscope 108, propagates along an optical channel within the arthroscope 108, and is eventually incident upon a capture array within the camera 110 (FIG. 1). The images detected by the capture array within the camera 110 are sent electronically to the surgical controller 118 (FIG. 1) and displayed on the display device 114 (FIG. 1). In one example, the arthroscope 108 is monocular or has a single optical path through the arthroscope for capturing images of the surgical site, notwithstanding that the single optical path may be constructed of two or more optical members (e.g., glass rods, optical fibers, etc.). That is to say, in example systems and methods the computer-assisted navigation provided by the arthroscope 108, the camera 110, and the surgical controller 118 is provided with the arthroscope 108 that is not a stereoscopic endoscope having two distinct optical paths separated by an interocular distance at the distal end endoscope.

During a surgical procedure, a surgeon selects an arthroscope with a viewing direction beneficial for the planned surgical procedure. Viewing direction refers to a line residing at the center of an angle subtended by the outside edges or peripheral edges of the view of an endoscope. The viewing direction for some arthroscopes is aligned with the longitudinal central axis of the arthroscope, and such arthroscopes are referred to as “zero degree” arthroscopes (e.g., the angle between the viewing direction and the longitudinal central axis of the arthroscope is zero degrees). The viewing direction of other arthroscopes forms a non-zero angle with the longitudinal central axis of the arthroscope. For example, for a 30º arthroscope the viewing direction forms a 30° angle to the longitudinal central axis of the arthroscope, the angle measured as an obtuse angle beyond the distal end of the arthroscope. In many cases for ACL repair, the surgeon selects a 30° arthroscope or a 45° arthroscope based on location the port created through the skin of the patient. In the example of FIG. 2, the view angle 210 of the arthroscope 108 forms a non-zero angle to the longitudinal central axis 212 of the arthroscope 108.

Still referring to FIG. 2, within the view of the arthroscope 108 is a portion of the bone 200 (e.g., within the intercondylar notch), along with the example bone fiducial 128 and the example touch probe 124. The example bone fiducial 128 is a multi-faceted element, with each face or facet having a fiducial disposed or created thereon. However, the bone fiducial need not have multiple faces, and in fact may take any shape so long as that shape, a fiducial or other visual marker, etc. can be tracked within the video images. The bone fiducial, such as bone fiducial 128, may be attached to the bone 200 in any suitable form. In this example, the bone fiducial is 128 fastened by a screw portion (not visible in FIG. 2, but visible in FIG. 1). The patterns of the fiducials on each facet are designed to provide information regarding the position and orientation of the bone fiducial 128 in the three-dimensional coordinate space of the view of the arthroscope 108. More particularly, the pattern is selected such that the position and orientation of the bone fiducial 128 may be determined from images captured by the arthroscope 108 and attached camera (FIG. 1).

The touch probe 124 is also shown as partially visible within the view of the arthroscope 108. The touch probe 124 may be used, as discussed more below, to identify a plurality of surface features on the bone 200 as part of the registration of the bone 200 to the three-dimensional bone model. Alternatively, though not specifically shown, the aimer 126 (FIG. 1) may be used as the device to assist with the registration process. In some cases, the touch probe 124 and/or the aimer 126 may carry their own, unique fiducials, such that their respective poses may be calculated from the one or more fiducials present in the video stream. However, in other cases, and as shown, the medical instrument used to help with registration of the three-dimensional bone model, be it the touch probe 124, the aimer 126, or any other suitable medical device, may omit carrying fiducials. Stated otherwise, in such examples the medical instrument has no fiducial markers/markings. In such cases, the pose of the medical instrument may be determined by a machine learning model, discussed in more detail below.

The images captured by the arthroscope 108 and attached camera are subject to optical distortion in many forms. For example, the visual field between a distal end of the arthroscope 108 and the bone 200 within the surgical site is filled with fluid, such as bodily fluids and saline used to distend the joint. Many arthroscopes have one or more lenses at the distal end that widen the field of view, and the wider field of view causes a “fish eye” effect in the captured images. Further, the optical elements within the arthroscope (e.g., rod lenses) may have optical aberrations inherent to the manufacturing and/or assembly process. Further still, the camera may have various optical elements for focusing the images received onto the capture array, and the various optical elements may have aberrations inherent to the manufacturing and/or assembly process. In example systems, prior to use within each surgical procedure, the endoscopic optical system is calibrated to account for the various optical distortions. The calibration creates a characterization function that characterizes the optical distortion, and further analysis of the frames of the video stream may be, prior to further analysis, compensated using the characterization function.

The next example step in the intraoperative procedure is the registration of the bone model created during the planning stage. During the intraoperative repair, the three-dimensional bone model is obtained by or provided to the surgical controller 118. Again using the example of anterior cruciate ligament repair, and specifically computer-assisted navigation for tunnel paths through the femur, the three-dimensional bone model of the lower portion of the femur is obtained by or provided to the surgical controller 118. Thus, the surgical controller 118 receives the three-dimensional bone model, and assuming the arthroscope 108 is inserted into the knee by way of a port through the patient’s skin, the surgical controller 118 also receives video images of a portion of the lower end of the femur. In order to relate the three-dimensional bone model to the images received by way of the arthroscope 108 and camera 110, the surgical controller 118 registers the three-dimensional bone model to the images of the femur received by way of the arthroscope 108 and camera 110.

In order to perform the registration, and in accordance with example methods, the bone fiducial 128 is attached to the femur. The bone fiducial placement is such that the bone fiducial is within the field of view of the arthroscope 108, but in a location spaced apart from the expected tunnel entry/exit point through the lateral condyle. More particularly, in example cases the bone fiducial 128 is placed within the intercondylar notch superior to the expected location of the tunnel through lateral condyle. To relate or register bone visible in the video images to the three-dimensional bone model, the surgical controller 118 (FIG. 1) is provided or determines a plurality of surface features of an outer surface of the bone. Identifying the surface features may take several forms, including a touch-based registration using the touch probe 124 without a carried fiducial, a touchless registration technique in which the surface features are identified after resolving the motion of the arthroscope 108 and camera relative to the bone fiducial 128, and a third technique in which uses a patient-specific instrument.

In the example touch-based registration, the surgeon may touch a plurality of locations using the touch probe 124 (FIG. 1). In some cases, particularly when portions of the outer surface of the bone are exposed to view, receiving the plurality of surface features of the outer surface of the bone may involve the surgeon “painting” the outer surface of the bone. “Painting” is a term of art that does not involve application of color or pigment, but instead implies motion of the touch probe 124 when the distal end of the touch probe 124 is touching bone. In this example, the touch probe 124 does not carry or have a fiducial visible to the arthroscope 108 and the camera 110. It follows that the pose of the touch probe 124 and the location of the distal tip of the touch probe 124 needs to be determined in order to gather the surface features for purposes of registering the three-dimensional bone model.

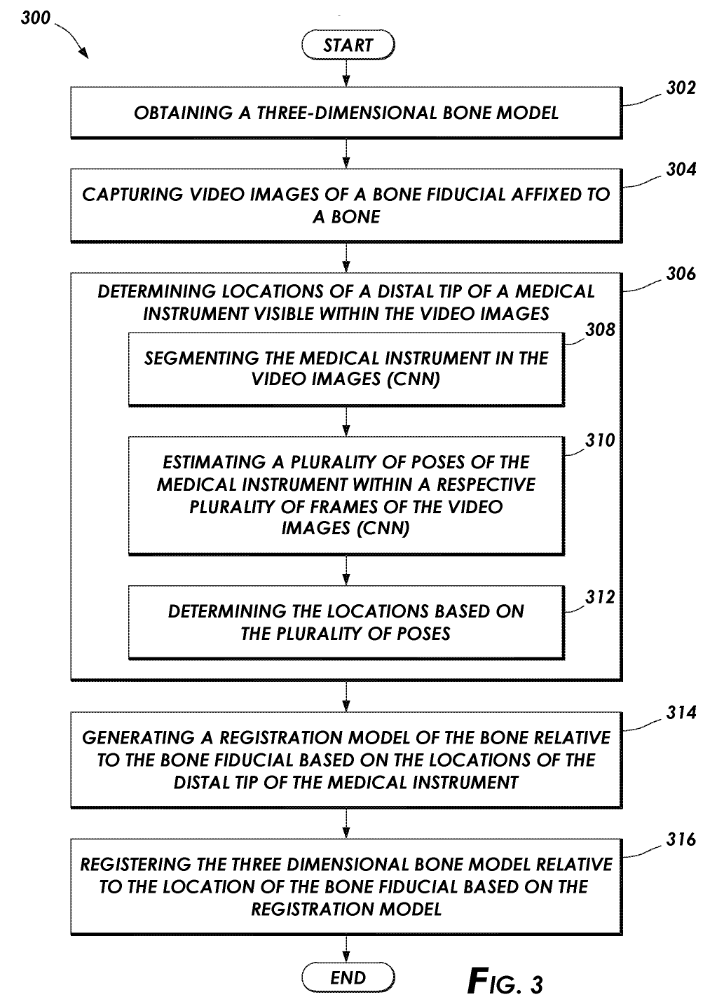

FIG. 3 shows a method 300 in accordance with at least some embodiments. The example method 300 may be implemented in software within a computer system, such as the surgical controller 118. In particular, the example method 300 comprises obtaining a three-dimensional bone model (block 302). That is to say, in the example method 300, what is obtained is the three-dimensional bone model that may be created by segmenting a plurality of non-invasive images (e.g., CT, MRI) taken preoperatively or intraoperatively. With the bone segmented from or within the images, the three-dimensional bone model may be created. The three-dimensional bone may take any suitable form, such as a computer-aided design (CAD) model, a point cloud of data points with respect to an arbitrary origin, or a parametric representation of a surface expressed using analytical mathematical equations. Thus, the three-dimensional bone model is defined with respect to the origin and in any suitable an orthogonal basis.

The next step in the example method 300 is capturing video images of the bone fiducial attached to the bone (block 304). The capturing is performed intraoperatively. In the example case of an arthroscopic anterior cruciate ligament repair, the capturing of video images is by way of the arthroscope 108 and camera 110. Other endoscopes may be used, such as endoscopes in which the capture array resides at the distal end of the device (e.g., chip-on-the-tip devices). However, in open procedures where the skin is cut and pulled away, exposing the bone to the open air, the capturing may be by any suitable camera device, such as one or both cameras of a stereoscopic camera system, or a portable computing device, such as a tablet or smart-phone device. The video images may be provided to the surgical controller 118 in any suitable form.

The next step in the example method 300 is determining locations of a distal tip of the medical instrument visible within the video images (block 306), where the distal tip is touching the bone in at least some of the frames of the video images, and the medical instrument does not have a fiducial. Determining the locations of the distal tip of the medical instrument may take any suitable form. In one example, determining the locations may include segmenting the medical instrument in the frames of the video images (block 308). The segmenting may take any suitable form, such as applying the video images to a segmentation machine learning algorithm. The segmentation machine learning algorithm may take any suitable form, such as neural network or convolution neural network trained with a training data set showing the medical instrument in a plurality of known orientations. The segmentation machine learning algorithm may produce segmented video images where the medical instrument is identified or highlighted in some way (e.g., box, brightness increased, a highlighted outline, other objects removed, etc.).

With the segmented video images, the example method 300 may estimate a plurality of poses of the medical instrument within a respective plurality of frames of the video images (block 310). The estimating the poses may take any suitable form, such as applying the video images to a pose machine learning algorithm. The pose machine learning algorithm may take any suitable form, such as neural network or convolution neural network trained to perform six-dimensional pose estimation. The resultant of the pose machine learning algorithm may be, for at least some of the frames of the video image, an estimated pose of the medical instrument in the reference frame of the video images and/or in the reference frame provided by the bone fiducial. That is, the result of the pose machine learning algorithm may be a plurality of poses, one pose each for at least some of the frames of the segmented video images. While in many cases a pose may be determined for each frame, in other cases it may not be possible to make a pose estimation for at least some frame because of video quality issues, such as motion blur caused by electronic shutter operation.

The next step in the example method 300 is determining the locations based on the plurality of poses (block 312). In particular, for each frame for which a pose can be estimated, based on a model of the medical device the location of the distal tip can be determined in the reference frame of the video images and/or the bone fiducial. Thus, the resultant is a set of locations that, at least some of which, represent locations of the outer surface of the bone.

FIG. 3 shows an example three-step process for determining the locations of the distal tip of the medical instrument. However, the method 300 is merely an example, and many variations are possible. For example, a single machine learning model, such as a convolution neural network, may be set up and trained to perform all three steps as a single overall process, though there may be many hidden layers of the convolution neural network. That is, the convolution neural network may segment the medical instrument, perform the six-dimensional pose estimation, and determine the location of the distal tip in each frame. The training data set in such a situation would include a data set in which each frame has the medical device segmented, the six-dimensional pose identified, and the location of the distal tip identified. The output of the determining step 306 may be a segmented video stream distinct from the video images captured at step 304. In such cases, the later method steps may use both segmented video stream and the video images to perform the further tasks. In other cases, the location information may be combined with the video images, such as being embedded in the video images, or added as metadata to each frame of the video images.

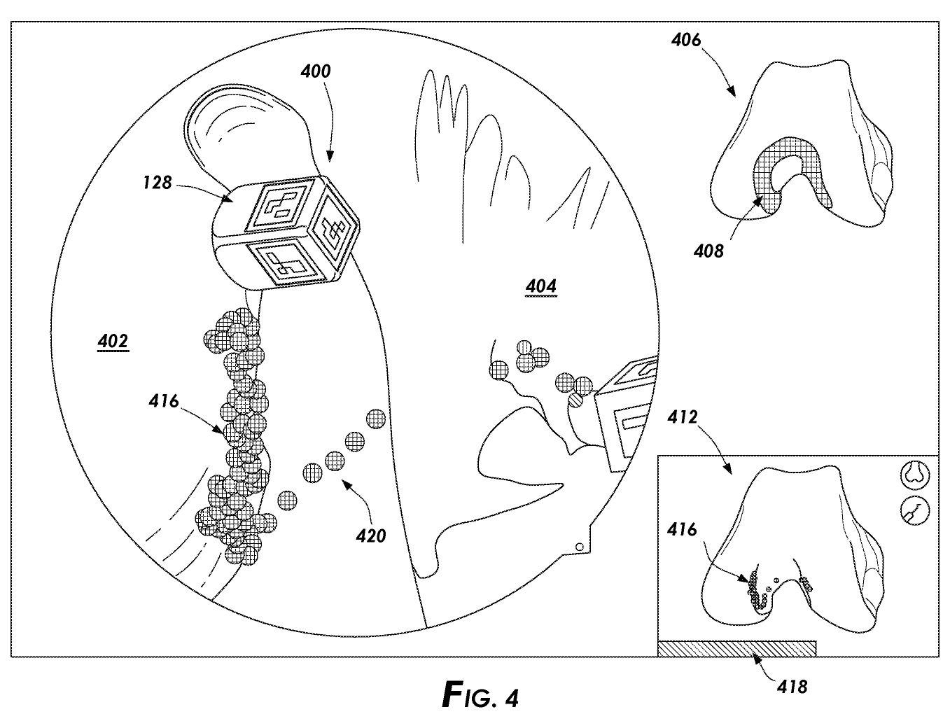

FIG. 4 is an example video display showing portions of a femur and a bone fiducial during a registration procedure. The display may be shown, for example, on the display device 114 associated with the device cart 102, or any other suitable location. In particular, visible in the main part of the display of FIG. 4 is an intercondylar notch 400, a portion of the lateral condyle 402, a portion the medial condyle 404, and the example bone fiducial 128. Shown in the upper right corner of the example display is a depiction of the bone, which may be a rendering 406 of the bone created from the three-dimensional bone model. Shown on the rendering 406 is a recommended area 408, the recommended area 408 being portions of the surface of the bone to be “painted” as part of the registration process. Shown in the lower right corner of the example display is a depiction of the bone, which again may be a rendering 412 of the bone created from the three-dimensional bone model. Shown on the rendering 412 are a plurality of surface features 416 on the bone model that have been identified as part of the registration process. Further shown in the lower right corner of the example display is progress indicator 418, showing the progress of providing and receiving of locations on the bone. The example progress indicator 418 is a horizontal bar having a length that is proportional to a level of confidence that the registration is accurate, but any suitable graphic or numerical display showing progress may be used (e.g., 0% to 100%).

Referring to both the main display and the lower right rendering, as the surgeon touches the outer surface of the bone within the images captured by the arthroscope 108 and camera 110, the surgical controller 118 receives the surface features on the bone, and may display each location both within the main display as dots or locations 416, and within the rendering shown in the lower right corner. More specifically, the example surgical controller 118 overlays indications of identified surface features 416 on the display of the images captured by the arthroscope 108 and camera 110, and in the example case shown, also overlays indications of identified surface features 416 on the rendering 412 of the bone model. Moreover, as the number of identified locations 416 increases, the surgical controller 118 also updates the progress indicator 418.

Returning to FIG. 3, the plurality of surface features 416 may be, or the example surgical controller 118 may generate, a registration model relative to the bone fiducial 128 (block 314). The registration model may take any suitable form, such as a computer-aided design (CAD) model or point cloud of data points in any suitable orthogonal basis. The registration model, regardless of the form, may have fewer overall data points or less “structure” than the bone model created by the non-invasive computer imaging (e.g., MRI). However, the goal of the registration model is to provide the basis for the coordinate transforms and scaling used to correlate the bone model to the registration model and relative to the bone fiducial 128. Thus, the next step in the example method 300 is registering the bone model relative to the location of the bone fiducial based on the registration model (block 316). Registration may conceptually involve testing a plurality of coordinate transformations and scaling values to find a correlation that has a sufficiently high correlation or confidence factor. Once a correlation is found with the sufficiently high confidence factor, the bone model is said to be registered to the location of the bone fiducial. Thereafter, the example registration method 300 may end; however, the surgical controller 118 may then use the registered bone model to provide computer-assisted navigation regarding a procedure involving the bone.

In the examples discussed to this point, registration of the bone model involves a touch-based registration technique using the touch probe 124 without a carried fiducial. However, other registration techniques are possible, such as a touchless registration technique. The example touchless registration technique again relies on placement of the bone fiducial 128. As before, when the viewing direction of the arthroscope 108 is relatively constant, the bone fiducial may have fewer faces with respective fiducials. Once placed, the bone fiducial 128 represents a fixed location on the outer surface of the bone in the view of the arthroscope 108, even as the position of the arthroscope 108 is moved and changed relative to the bone fiducial 128. Again, in order to relate or register the bone visible in the video images to the three-dimensional bone model, the surgical controller 118 (FIG. 1) determines a plurality of surface features of an outer surface of the bone, and in this example determining the plurality of surface features is based on a touchless registration technique in which the surface features are identified based on motion of the arthroscope 108 and camera 110 relative to the bone fiducial 128.

Another technique for registering the bone model to the bone uses a patient-specific instrument. In both touch-based and touchless registration techniques, a registration model is created, and the registration model is used to register the bone model to the bone visible in the video images. Conceptually, the registration model is used to determine a coordinate transformation and scaling to align the bone model to the actual bone. However, if the orientation of the bone in the video images is known or can be determined, use of the registration model may be omitted, and instead the coordinate transformations and scaling may be calculated directly.

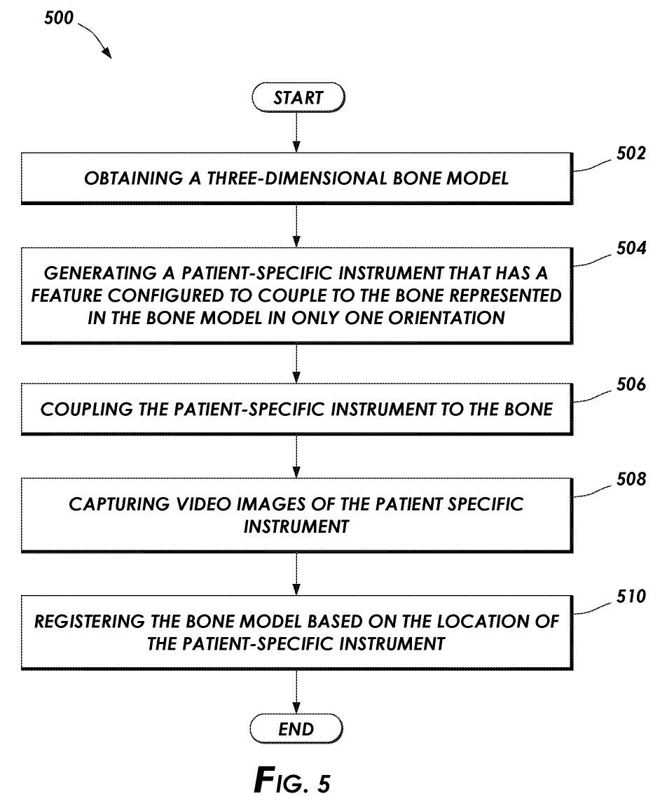

FIG. 5 shows a method 500 in accordance with at least some embodiments. The example method may be implemented in software within one or more computer systems, such as, in part, the surgical controller 118. In particular, the example method 500 starts and comprises obtaining a three-dimensional bone model (block 502). Much like the prior techniques, in the patient-specific instrument registration technique what is obtained is the three-dimensional bone model that may be created by segmenting a plurality of non-invasive images (e.g., MRI) taken preoperatively or intraoperatively.

The next step in the example method 500 is generating a patient-specific instrument that has a feature designed to couple to the bone represented in the bone model in only one orientation (block 504). Generating the patient-specific instrument may first involve selecting a location at which the patient-specific instrument will attach. For example, a device or computer system may analyze the bone model and select the attachment location. In various examples, the attachment location may be a unique location in the sense that, if a patient-specific instrument is made to couple to the unique location, the patient-specific instrument will not couple to the bone at any other location. In the example case of an anterior cruciate ligament repair, the location selected may be at or near the upper or superior portion on the intercondylar notch. If the bone model shows another location with a unique feature, such as a bone spur or other raised or sunken surface anomaly, such a unique location may be selected as the attachment location for the patient-specific instrument.

Moreover, forming the patient-specific instrument may take any suitable form. In one example, a device or computer system may directly print, such as using a 3D printer, the patient-specific instrument. In other cases, the device or computer system may print a model of the attachment location, and the model may then become the mold for creating the patient-specific instrument. For example, the model may be the mold for an injection-molded plastic or casting technique. In some examples, the patient-specific instrument carries one or more fiducials, but as mentioned above, in other cases the patient-specific instrument may itself be tracked and thus carry no fiducials.

The next step in the example method 500 is coupling the patient-specific instrument to the bone, in some cases the patient-specific instrument having the fiducial coupled to an exterior surface (block 506). As previously mentioned, the attachment location for the patient-specific instrument is selected to be unique such that the patient-specific instrument couples to the bone in only one location and in only one orientation. In the example case of an arthroscopic ACL repair, the patient-specific instrument may be inserted arthroscopically. That is, the attachment location may be selected such that a physical size of the patient-specific instrument enables insertion through the ports in the patient’s skin. In other case, the patient-specific instrument may be made or constructed of a flexible material that enables the patient-specific instrument to deform for insertion in the surgical site, yet return to the predetermined shape for coupling to the attachment location. However, in open procedures where the skin is cut and pulled away, exposing the bone to the open air, the patient-specific instrument may be a rigid device with fewer size restrictions.

The next step in the example method 500 is capturing video images of the patient-specific instrument (block 508). Here again, the capturing may be performed intraoperatively. In the example case of an arthroscopic anterior cruciate ligament repair, the capturing of video images is by the surgical controller 118 by way of arthroscope 108 and camera 110. However, in open procedures where the skin is cut and pulled away, exposing the bone to the open air, the capturing may be by any suitable camera device, such as one or both cameras of a stereoscopic camera systems, or a portable computing device, such as a tablet or smart-phone device. In such cases, the video images may be provided to the surgical controller 118 in any suitable form.

The next step in the example method 500 is registering the bone model based on the location of the patient-specific instrument (block 510). That is, given that the patient-specific instrument couples to the bone at only one location and in only one orientation, the location and orientation of the patient-specific instrument is directly related to the location and origination of the bone, and thus the coordinate transformations and scaling for the registration may be calculated directly. Thereafter, the example method 500 may end; however, the surgical controller 118 may then use the registered bone model to provide computer-assisted navigation regarding a surgical task or surgical procedure involving the bone.

For example, with the registered bone model the surgical controller 118 may provide guidance regarding a surgical task of a surgical procedure. The specific guidance is dependent upon the surgical procedure being performed and the stage of the surgical procedure. A non-exhaustive list of guidance comprises: changing a drill path entry point; changing a drill path exit point; aligning an aimer along a planned drill path; showing location at which to cut and/or resect the bone; reaming the bone by a certain depth along a certain direction; placing a device (suture, anchor or other) at a certain location; placing a suture at a certain location; placing an anchor at a certain location; showing regions of the bone to touch and/or avoid; and identifying regions and/or landmarks of the anatomy. In yet still other cases, the guidance may include highlighting within a version of the video images displayed on a display device, which can be the arthroscopic display or a see-through display, or by communicating to a virtual reality device or a robotic tool.

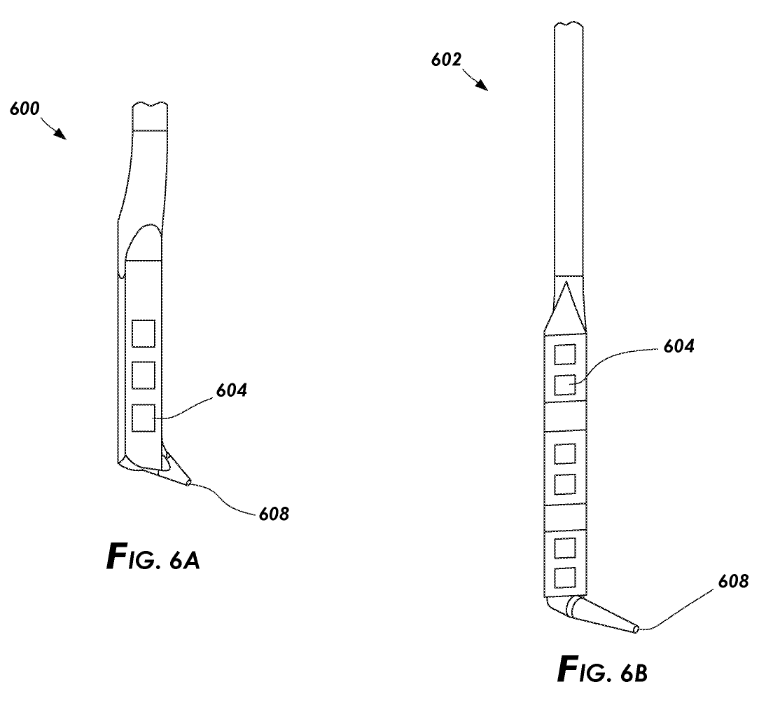

In these and other examples of a registration procedure, a user probes an anatomical surface using a handheld probe. FIGS. 6A and 6B show example probes 600 and 602, respectively. In these examples, the probes 600, 602 include one or more fiducials or fiducial markers 604. Tips 608 of the probes 600, 602 can be calibrated such that locations of the tips 608 relative to the fiducials 604 are known. For example, the probes 600, 602 are calibrated, in a pre-operative calibration procedure, to obtain geometrical and dimensional data such that position or location, orientation, etc. of various features of the probes 600, 602, such as the tips 608, relative to the fiducials 604 are known. In this manner, 3D points can be intra-operatively reconstructed in the reference frame of the fiducial marker. For example, points can be obtained when the tip is in contact with the surface of patient anatomy (by determining the location of the tip based on the detected fiducials 604). Accordingly, the set of reconstructed 3D points is a representation of the anatomy of interest that is provided as input to the registration procedure.

Surgical navigation systems and methods according to the present disclosure are configured to detect and track a surgical instrument, such as an awl, as the awl is moved within a surgical environment. For example, the awl may include one or more fiducial markers. A camera is used to detect the fiducial markers and track movement/position of the awl within the surgical environment (e.g., relative to or within a coordinate plane of a fixed fiducial/bone marker or other point of reference). In this manner, the awl can be tracked while navigating between and/or designating various points on patient anatomy in accordance with various settings and guidance provided by the surgical navigation system. For example, while navigating the surgical environment with the awl, various inputs are provided to identify anatomical boundaries or landmarks within the surgical environment and navigation guidance (e.g., visual guidance for a target tunnel position) is provided based on the inputs and various other operational parameters/settings. In some examples, the awl can be used to form an indentation or other feature in the bone, such as by using a mallet or other tool to apply force to the awl.

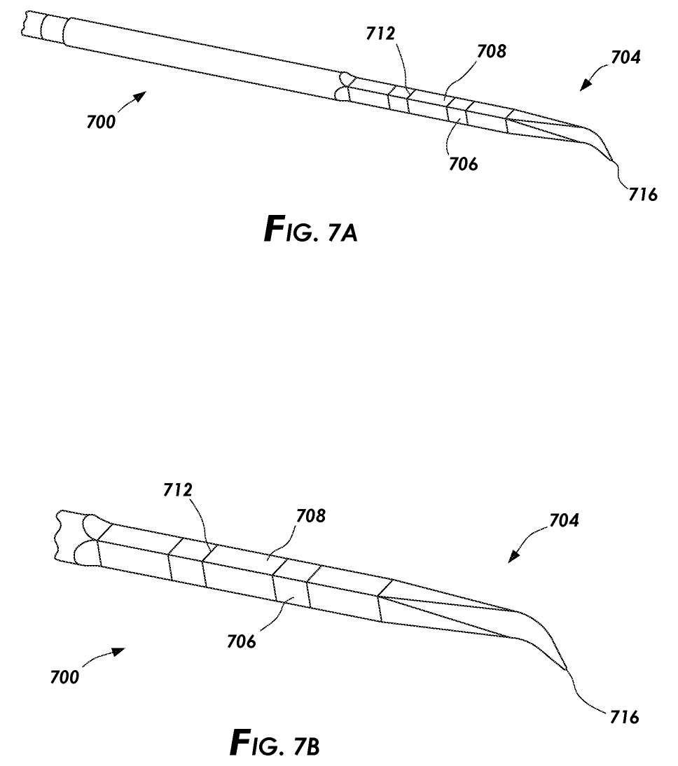

FIGS. 7A and 7B show an example awl 700 according to the principles of the present disclosure. FIG. 7B is a close-up illustration of a tip 704 of the awl 700. As shown, the tip 704 has a triangular, square, rectangular, etc. shape or profile defining one or more planar faces/surfaces 706. The tip 704 of the awl 700 includes one or more fiducial markers 708. For example, the tip 704 may include one or more banded sections 712 (e.g., three of the banded sections 712, as shown). Each of the banded sections 712 may include one or more fiducial markers 708 (e.g., one or more of the fiducial markers 708 on each of the planar faces 706. In this manner, the position, orientation, and movement of the awl 700 can be tracked by detecting the fiducial markers 708 as described above. Various points on patient anatomy can be selected based on a point indicated by a pointer tip 716 of the awl 700.

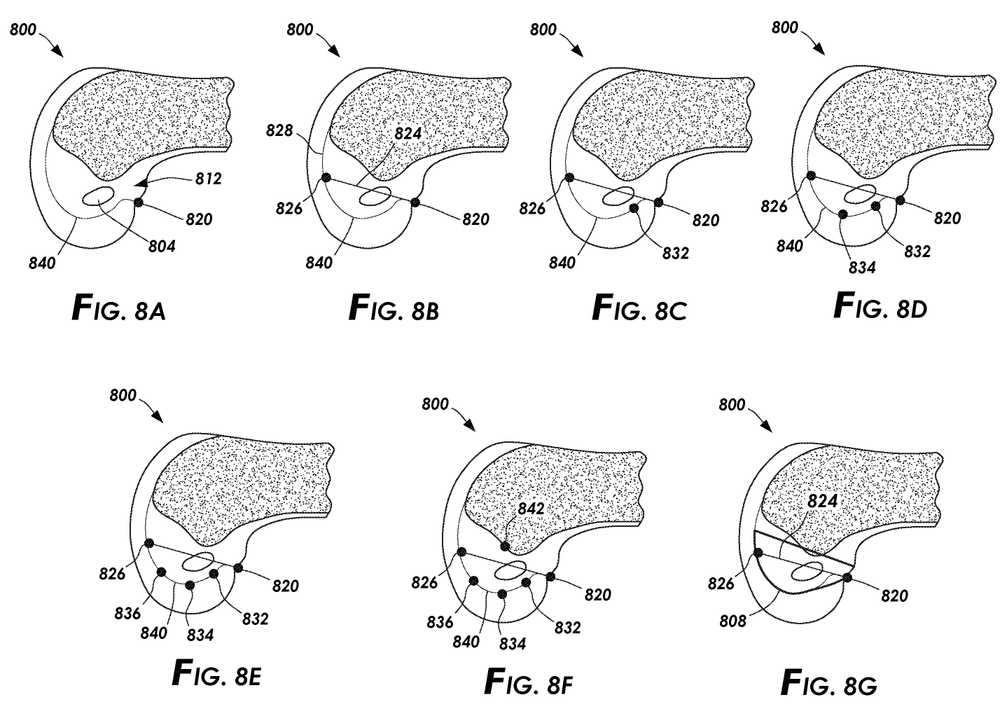

FIGS. 8A, 8B, 8C, 8D, 8E, 8F, and 8G show an example workflow/navigation process performed to select/identify points on patient anatomy (e.g., a femur 800, as shown with an example femoral ACL attachment point/region 804) for tunnel positioning according to the principles of the present disclosure. For example, an instrument, such as the awl 700, is used to select a desired bounding region 808 for an ACL footprint. More specifically, the awl 700 is used to select/identify a plurality of bounding points on a same wall of the femur 800 as a femoral condyle 812. Selecting the bounding points may include, for example, providing one or more inputs to the surgical navigation system (e.g., by pressing a button on an instrument, computing device, etc. or otherwise providing an input via an interface coupled to the surgical navigation system) while the pointer tip 716 of the awl 700 is pointing to a desired/intending point on the surface of the femur 800. In some examples, selecting the bounding points may include forming an indentation or other feature in the bone, such as by using a mallet or other tool to apply force to the awl 700.

In this example, as shown in FIG. 8A, the user selects the location of the femoral condyle 804 by selecting a point 820, which may correspond to a posterior reference point. In other words, distances of various points from a back wall of the femoral condyle 804 (a back wall distance) can be measured based on respective distances from the posterior reference point 820.

As shown in FIG. 8B, the user identifies/defines a long axis 824 of the femoral condyle 804 by selecting an anterior-most point 826 of the condylar wall 828 at the point of curvature. The point 826, along with the posterior reference point 820, defines a distance that can be used to determine/define a percentage (e.g., a distance percentage) of a point along a condyle length. In other words, a distance percentage for a given point on the axis 824 corresponds to a percentage of an overall distance, along the condyle length, from the posterior reference point 820 to the point 826. As one example, a midpoint on the axis 824 between the point 820 and the point 826 would have a distance percentage of 50%. The distance percentage may correspond to the distance percentage of a center point of a tunnel or planned tunnel entry point.

As shown in FIGS. 8C, 8D, and 8E, with the long axis 824 established, the user selects various points 832, 834, and 836 along a surface of an inferior condyle wall 840 from the point 820 to the point 826 (e.g., from back to front within the joint) to define a lower boundary for placing a tunnel.

As shown in FIG. 8F, the user selects a point 842, which defines a superior-most point (e.g., a highest point permitted for a femoral footprint location) of the bounding region 808.

As shown in FIG. 8G, the selected points define the bounding region 808, which corresponds to a bounding plane upon which a femoral tunnel circle can be projected and bounded by the surgical navigation system. In other words, in accordance with the points selected by the user and detected/analyzed by the surgical navigation system, the surgical navigation system is configured to determine/calculate and project (e.g., display, as visual guidance) the bounding region 808, and a tunnel location/position can be displayed relative to the bounding region 808.

Although described above with respect to selection of the points 820, 826, 832, 834, 836, and 842, in other examples fewer or more points can be selected and used to define the bounding region/plane 808. In other examples, rather than selecting discrete/individual points, the awl can be used to trace/point an outline of the bounding region 808 (e.g., to perform continual point collection).

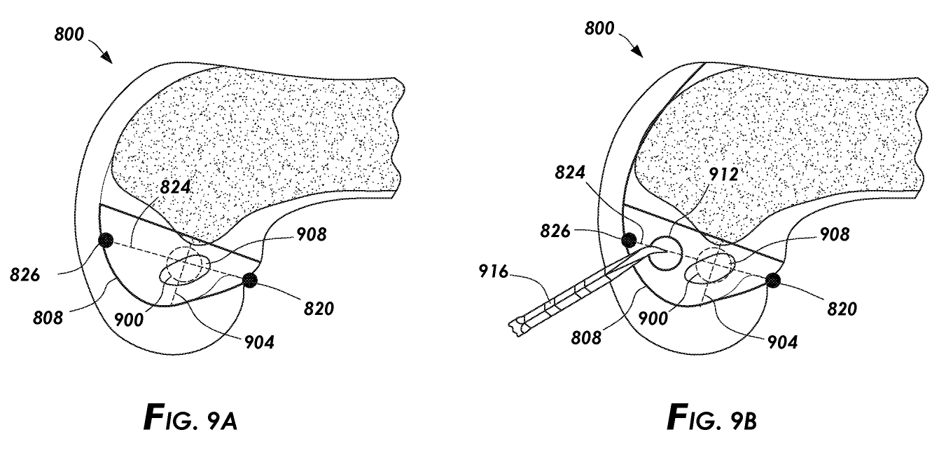

For example, FIGS. 9A and 9B show example projections/visual guidance provided by the surgical navigation system of the present disclosure, in accordance with data/points collected as the user indicates various points on the patient anatomy as described above in FIGS. 8A-8G. For example, as shown in FIG. 9A, with the bounding plane established (i.e., as defined by the bounding region 808), a target tunnel location 900 can be displayed at a target distance percentage (e.g., corresponding to a center point of the tunnel/tunnel location 900). For example, a target percentage line 904 can be displayed at a target percentage (e.g., a target distance percentage input by the user, calculated or stored by the surgical navigation system, etc.) along and perpendicular to the long axis 824.

In some examples, a target back wall distance line 908 can be displayed. The target back wall distance line 908 indicates a target back wall distance (i.e., a target distance from the back wall of the femur, such as defined by the posterior reference point 820 to an edge/wall of the tunnel), which may be determined in accordance with user inputs/settings/parameters (e.g., an input target back wall distance). As an example, the line 908 indicating back wall distance may correspond to a line tangent to the circle (i.e., the tunnel location 900) and perpendicular to the long axis 824.

As shown in FIG. 9B, a targeted tunnel footprint 912 can be displayed in real-time as the user moves an awl (e.g., which can be detected and displayed/rendered as a virtual awl 916 using the techniques described herein) within the surgical site. In other words, as the user moves the awl, the location of the targeted tunnel footprint 912 is displayed in updated in real-time, providing guidance to the user until the targeted tunnel footprint 912 is in a suitable location (e.g., in accordance with the tunnel location 900, the target percentage line 904, the target back wall distance line 908, etc.). When the user finds a desired/suitable location for the tunnel, the awl can be struck to mark the bone with an indentation and a guide pin can be placed based on the indentation. In this manner, the surgical navigation system is configured to provide visual guidance for determining tunnel position based on settings, user inputs, and data collected in accordance with user identification/selection of various points on patient anatomy.

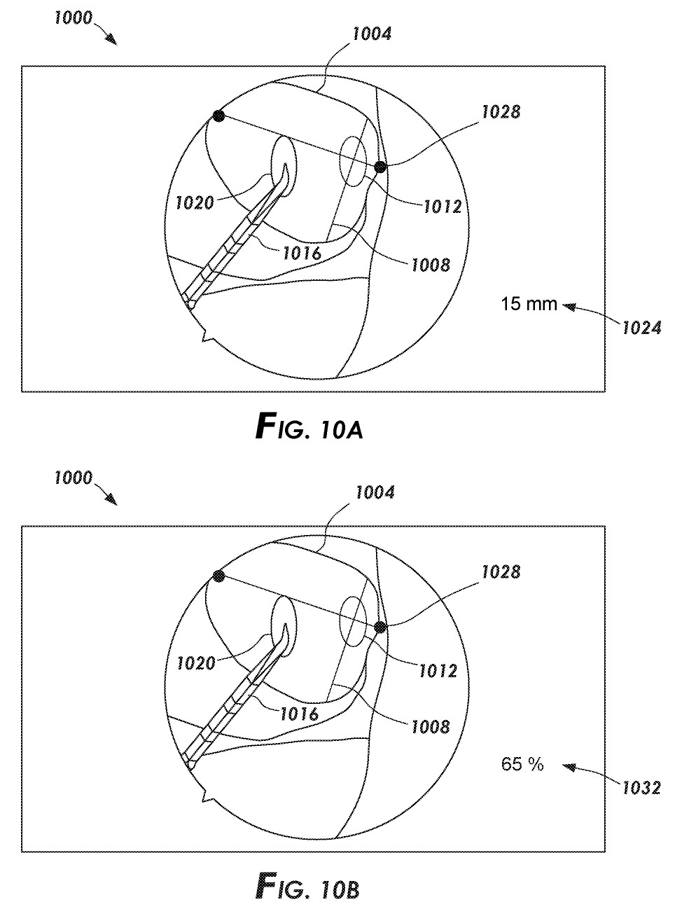

FIGS. 10A and 10B show examples of visual guidance displayed to assist a user to locate a tunnel. In some examples, the visual guidance includes one or more constraints associated with tunnel location/position. As shown in FIG. 10A, an example display 1000 of the surgical site (e.g. a real-time video captured by the camera as viewed through an arthroscope) includes a bounding region 1004, a target percentage line 1008, an example target tunnel location 1012, a virtual/digital rendering of an awl 1016, and a targeted tunnel footprint 1020 aligned with a tip of the awl 1016. In this example, the display 1000 includes a real-time back wall distance/measurement as shown at 1024 (e.g., a distance from an edge/wall of the footprint 1020 and a back wall 1028.

The back wall distance 1024 is updated in real-time (e.g., increased and decreased) as the user moves the awl 1016 within the surgical site. The display 1000 may include a visual indication of whether the footprint 1020 is in a position/location that satisfies one or more conditions/thresholds, such as a desired (e.g., minimum) back wall distance 1024. For example, the text/font for the back wall distance 1024 may have a first characteristic (e.g., color, such as red or yellow) when the back wall distance 1024 is not within a predetermined range of a desired/target back wall distance 1024. Conversely, the text/font for the back wall distance 1024 may have a second characteristic when the back wall distance 1024 is within the predetermined range of a desired/target back wall distance 1024 (e.g., the font may change from yellow to green). Other types of visual (or audio) indicators may be used to alert the user that the back wall distance 1024 is outside of the predetermined range, below a threshold, etc.

FIG. 10B shows another example of the display 1000. In this example, the display 1000 includes a real-time distance percentage as shown at 1032. Similar to the above example in FIG. 10A, the text/font for the distance percentage 1032 can by modified to indicate whether the distance percentage of the footprint 1020 is within a predetermined range (e.g., within 3% of) of the target percentage line 1008. In various examples, one or both of the back wall distance 1024 and the distance percentage 1032 can be displayed, and/or other types of visual guidance may be displayed.

As an example, settings input to (e.g., via a user interface) or otherwise obtained by the surgical navigation system may include femoral entry point tunnel diameter (e.g., in 1.0 mm increments), target back wall thickness/distance (e.g., in 1.0 mm increments) and/or back wall distance percentage, offset of the tunnel wall edge from the back wall, target percentage of tunnel center along an anterior-posterior axis of the femoral condyle, display toggle for various visual guidance elements (e.g., a bounding plane that limits location of the ACL tunnel footprint, back wall distance, tunnel center, etc.), tunnel footprint shape, etc.

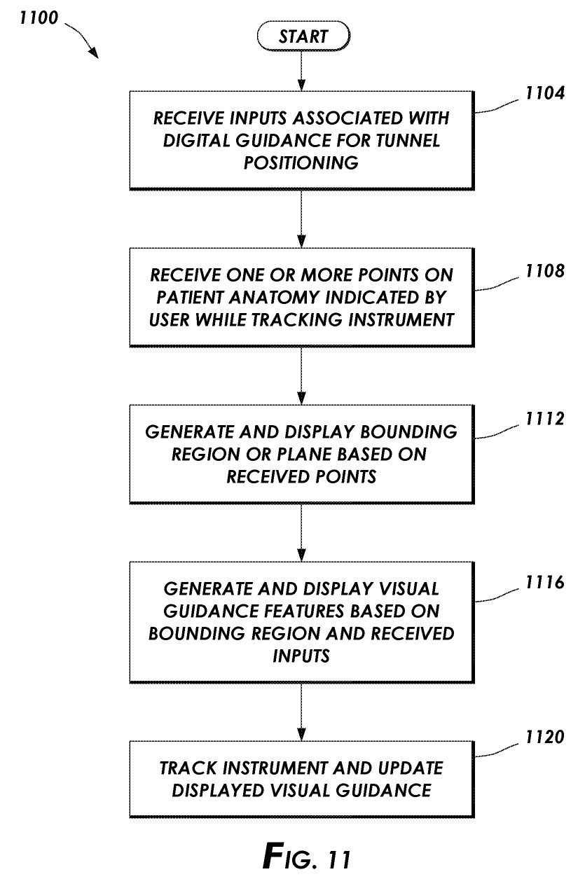

FIG. 11 illustrates steps of an example method 1100 for performing surgical navigation techniques in accordance with at least some embodiments. In an example, the method 1100 may include, or be performed subsequent to, performing a touch-based registration procedure using a probe. The method 1100 can be performed in conjunction with any of the other methods described herein. In an example, a computing device, one or more processors or processors, etc. are configured to perform the steps or functions of the method 1100.

At 1104, the method 1100 includes receiving one or more inputs or settings (e.g., user inputs, stored or calibrated settings, etc.) associated with visual guidance for tunnel positioning according to the principles of the present disclosure. The inputs/settings may correspond to various parameters for calculating and providing visual guidance as described herein, including, but not limited to, femoral entry point diameter, target back wall distance, back wall distance percentage, and so on.

At 1108, the method 1100 includes receiving one or more points on patient anatomy as indicated by a user (e.g., data indicative of points selected by the user) as a tool or instrument (e.g., an awl) is moved within a surgical site. For example, receiving the points includes, but is not limited to, while detecting the position of the instrument within the surgical site, receiving a posterior reference point, an anterior-most point, one or more points along a surface of an inferior condyle wall, a superior-most point, etc. At 1112, the method 1100 includes generating and displaying a bounding region/plane based on the received points. The bounding region may be displayed over a rendered/digital image of the surgical site, a real-time (live) image feed of the surgical site, etc.

At 1116, the method 1100 includes generating and displaying one or more visual guidance features based on the bounding region and the received inputs or settings. The visual guidance features may include, but are not limited to, a back wall distance, a target tunnel location, a long axis, a target distance percentage line, a distance percentage, etc.

At 1120, the method 1100 includes tracking the instrument as the instrument is moved within the surgical site and updating the displayed visual guidance based on the tracked instrument. For example, updating the visual guidance may include, but is not limited to, updating a displayed position/orientation of a digital/virtual rendering of the instrument and a corresponding targeted tunnel footprint, updating a back wall distance and/or back wall distance percentage, providing one or more indicators of whether the targeted tunnel footprint satisfies one or more ranges/thresholds specified in the received inputs or settings, etc.

In some examples, image-based tracking techniques such as simultaneous localization and mapping (SLAM) techniques instead of a fixed fiducial marker for pose estimation of the camera.

In some examples, anatomical features can be auto-detected to eliminate the need for tracing/point selection for defining the bounding region and determining an acceptable zone for the ACL footprint.

In some examples, additional points can be input to adjust the boundary region estimate more precisely by shifting a closest boundary point to match the location of the additional points.

In some examples, major and minor axes of a traced region can be automatically detected to determine an estimated location or function as a boundary for back wall or other calculations.

In some examples, the techniques described herein can be implemented with respect to other anatomy, such as the tibia, with an estimate based on a line drawn anterior to posterior to place a suggested location, or to provide safe zones for tibial crest and anterior wall blowouts. The techniques can also be applied to other joint spaces and procedures that could benefit from setting safe zones and boundaries.

In some examples, an alternative workflow could include placing a digital ACL footprint circle or oval using the tool (either freehand or using the line-estimation method), and then providing a closest distance measurement from tooltip to a bounding box of an augmented reality target. The user would then be able to place the probe along the condylar wall to check back wall distances at points of interest, or slide the probe along the condyle to quickly confirm that sufficient buffer zones exist. An indicator of whether the placed footprint location is in or out of the bounding plane can be provided to prevent incorrect estimates, and/or projection mathematics can be used to provide more accurate values.

In some examples, an awl or other instrument may include one or more input mechanisms (e.g., buttons or selectors) that allow the user to provide various inputs, such as to cycle through various preset inputs or settings (e.g., footprint shape, diameter, etc.).

In some examples, the techniques described herein can be implemented with other types of tools, such as a combination awl/aimer. In one example, an aimer includes an additional protrusion that can function as a probe and/or awl tip for point collection.

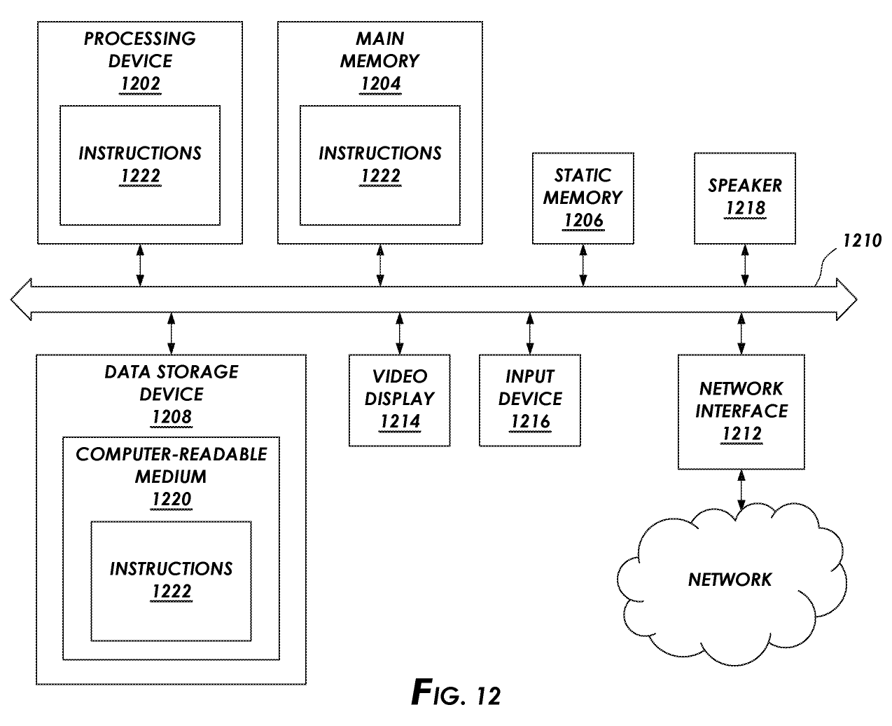

FIG. 12 shows an example computer system 1200. In one example, the computer system 1200 may correspond to the surgical controller 118, a tablet device within the surgical room, or any other system that implements any or all the various methods discussed in this specification. The computer system 1200 may be connected (e.g., networked) to other computer systems in a local-area network (LAN), an intranet, and/or an extranet (e.g., device cart 102 network), or at certain times the Internet (e.g., when not in use in a surgical procedure). The computer system 1200 may be a server, a personal computer (PC), a tablet computer or any device capable of executing a set of instructions (sequential or otherwise) that specify actions to be taken by that device. Further, while only a single computer system is illustrated, the term “computer” shall also be taken to include any collection of computers that individually or jointly execute a set (or multiple sets) of instructions to perform any one or more of the methods discussed herein.

The computer system 1200 includes a processing device 1202, a main memory 1204 (e.g., read-only memory (ROM), flash memory, dynamic random access memory (DRAM) such as synchronous DRAM (SDRAM)), a static memory 1206 (e.g., flash memory, static random access memory (SRAM)), and a data storage device 1208, which communicate with each other via a bus 1210.

Processing device 1202 represents one or more general-purpose processing devices such as a microprocessor, central processing unit, or the like. More particularly, the processing device 1202 may be a complex instruction set computing (CISC) microprocessor, reduced instruction set computing (RISC) microprocessor, very long instruction word (VLIW) microprocessor, or a processor implementing other instruction sets or processors implementing a combination of instruction sets. The processing device 1202 may also be one or more special-purpose processing devices such as an application specific integrated circuit (ASIC), a field programmable gate array (FPGA), a digital signal processor (DSP), network processor, or the like. The processing device 1202 is configured to execute instructions for performing any of the operations and steps discussed herein. Once programmed with specific instructions, the processing device 1202, and thus the entire computer system 1200, becomes a special-purpose device, such as the surgical controller 118.

The computer system 1200 may further include a network interface device 1212 for communicating with any suitable network (e.g., the device cart 102 network). The computer system 1200 also may include a video display 1214 (e.g., display device 114), one or more input devices 1216 (e.g., a microphone, a keyboard, and/or a mouse), and one or more speakers 1218. In one illustrative example, the video display 1214 and the input device(s) 1216 may be combined into a single component or device (e.g., an LCD touch screen).

The data storage device 1208 may include a computer-readable storage medium 1220 on which the instructions 1222 (e.g., implementing any methods and any functions performed by any device and/or component depicted described herein) embodying any one or more of the methodologies or functions described herein is stored. The instructions 1222 may also reside, completely or at least partially, within the main memory 1204 and/or within the processing device 1202 during execution thereof by the computer system 1200. As such, the main memory 1204 and the processing device 1202 also constitute computer-readable media. In certain cases, the instructions 1222 may further be transmitted or received over a network via the network interface device 1212.

While the computer-readable storage medium 1220 is shown in the illustrative examples to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any medium that is capable of storing, encoding or carrying a set of instructions for execution by the machine and that cause the machine to perform any one or more of the methodologies of the present disclosure. The term “computer-readable storage medium” shall accordingly be taken to include, but not be limited to, solid-state memories, optical media, and magnetic media.

The computer system 1200 or one or more computing or processing devices may be configured to perform functions of the procedures described herein, including functions related to communication and/or control of any of the instruments, functions, steps, etc. described in FIGS. 7-11, and/or functions of the methods described herein.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

What is claimed is:

1. A processor configured to execute instructions stored in memory to control a surgical navigation system, wherein executing the instructions causes the processor to control the surgical navigation system to:
receive one or more images of a surgical environment;
determine a plurality of points on patient anatomy indicated by an instrument identified in the surgical environment;
define a bounding plane on the patient anatomy based on the plurality of points; and
generate, for display, visual guidance based on the bounding plane and a location of the instrument, wherein the visual guidance includes (i) at least one constraint for a location of a tunnel to be formed in the patient anatomy, (ii) a visual indicator of the location of the tunnel based on the location of the instrument, and (iii) an indication of whether the location of the tunnel satisfies the at least one constraint.

2. The processor of claim 1, wherein the at least one constraint includes a distance from the location of the tunnel to a feature of the patient anatomy.

3. The processor of claim 2, wherein the distance is a distance to a back wall of a lateral femoral condyle.

4. The processor claim 1, wherein the at least one constraint includes a distance percentage along an axis defined by a posterior reference point on a lateral femoral condyle and an anterior-most point of a condylar wall.

5. The processor of claim 1, wherein the indication of whether the location of the tunnel satisfies the at least one constraint includes a characteristic of display of the at least one constraint.

6. The processor of claim 1, wherein executing the instructions causes the processor to control the surgical navigation system to:
receive one or more parameters; and
display the visual guidance based on the one or more parameters.

7. The processor of claim 6, wherein the one or more parameters include at least one of a femoral entry point tunnel diameter and a target back wall thickness.

8. The processor of claim 1, wherein the plurality of points includes at least one of a posterior reference point on a femoral condyle and an anterior-most point of a condylar wall.

9. The processor of claim 8, wherein the plurality of points further includes one or more points along a surface of an inferior condyle wall between the posterior reference point and the anterior-most point.

10. The processor of claim 9, wherein the plurality of points further includes a superior-most point of the bounding plane.

11. A method for controlling a surgical navigation system, the method comprising:
receiving one or more images of a surgical environment;
determining a plurality of points on patient anatomy indicated by an instrument identified in the surgical environment;
defining a bounding plane on the patient anatomy based on the plurality of points; and
generating, for display, visual guidance based on the bounding plane and a location of the instrument, wherein the visual guidance includes (i) at least one constraint for a location of a tunnel to be formed in the patient anatomy, (ii) a visual indicator of the location of the tunnel based on the location of the instrument, and (iii) an indication of whether the location of the tunnel satisfies the at least one constraint.

12. The method of claim 11, wherein the at least one constraint includes a distance from the location of the tunnel to a feature of the patient anatomy.

13. The method of claim 12, wherein the distance is a distance to a back wall of a lateral femoral condyle.

14. The method claim 11, wherein the at least one constraint includes a distance percentage along an axis defined by a posterior reference point on a lateral femoral condyle and an anterior-most point of a condylar wall.

15. The method of claim 11, wherein the indication of
whether the location of the tunnel satisfies the at least one constraint includes a characteristic of display of the at least one constraint.

16. The method of claim 11, further comprising:
receiving one or more parameters; and
displaying the visual guidance based on the one or more parameters.

17. The method of claim 16, wherein the one or more parameters include at least one of a femoral entry point tunnel diameter and a target back wall thickness.

18. The method of claim 11, wherein the plurality of points includes at least one of a posterior reference point on a femoral condyle and an anterior-most point of a condylar wall.

19. The method of claim 18, wherein the plurality of points further includes one or more points along a surface of an inferior condyle wall between the posterior reference point and the anterior-most point and a superior-most point of the bounding plane.

20. A surgical navigation system, comprising:
an image capture device configured to capture one or more images of a surgical environment; and
a controller configured to
detect a location of an instrument within the surgical environment using the one or more images and one or more fiducial markers on the instrument,
identify a plurality of points on patient anatomy based on respective locations of the instrument within the surgical environment,
define a bounding plane on the patient anatomy based on the plurality of points, and
generate, for display, visual guidance based on the bounding plane and the location of the instrument, wherein the visual guidance includes (i) at least one constraint for a location of a tunnel to be formed in the patient anatomy, (ii) a visual indicator of the location of the tunnel based on the location of the instrument, and (iii) an indication of whether the location of the tunnel satisfies the at least one constraint,
wherein the at least one constraint includes at least one of
a distance from an edge of the tunnel to a back wall of a lateral femoral condyle, and
a distance percentage along an axis defined by a posterior reference point on the lateral femoral condyle and an anterior-most point of a condylar wall.
